(19) United States
(12) Patent Application Publication
(10) Pub. No.: US 2008/0227638 A1
(43) Pub. Date: Sep. 18, 2008

(54) Use of abscisic acid to enhance growth control

(76) Inventors: Xiaozhong Liu, Vernon Hills, IL (US); Peter D. Petracek, Grayslake, IL (US); Daniel F. Heiman, Libertyville, IL (US); Mark Beach, Kenosha, WI (US); Prem Warrior, Green Oaks, IL (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 12/011,810

(22) Filed: Jan. 30, 2008

Related U.S. Application Data
(60) Provisional application No. 60/898,593, filed on Jan. 31, 2007.

(51) Int. Cl. A01N 37/06; C07C 63/64; A01P 21/00

(52) U.S. Cl. 504/142; 562/508

(57) ABSTRACT

This invention describes the use of S-(+)-abscisic acid (ABA) or its salts in combination with gibberellin biosynthesis inhibitors to improve the performance of gibberellin synthesis inhibitors, and to increase water conservation in plants such as turfgrass.

### FIELD OF THE INVENTION

The present invention is directed to improving the performance of gibberellin synthesis inhibitors by hastening growth control, providing additional growth control, extending the growth inhibitory effect of gibberellin inhibitors and increasing water conservation by using combinations of gibberellin synthesis inhibitors and abscisic acid or its salts.

### BACKGROUND OF THE INVENTION

Abscisic acid (ABA) is a natural plant growth regulator that is responsible for stress tolerance. ABA causes stomatal closure (Assmann, S. 2004 In: Plant Hormones Biosynthesis, Signal Transduction, Action ed. P. J. Davies, p 391-412). The stomatal closure caused by ABA can contribute to the reduction of plant transpiration and thus increase drought and water conservation. Although ABA has been shown to reduce plant growth (Petracek, P. D., D. Woolard, R. Menendez and P. Warrior, 2005, Proc. PGRSA, 32: 7-9), its effect on growth is less well understood.

Mowing is one of the major practices in turfgrass management. Turfgrass growth retardants, which are specifically referred to as turfgrass plant growth regulators (Turfgrass PGRs or Turf PGRs), have been widely used by the turfgrass industry to suppress growth and thus to reduce mowing frequency and clippings. Turfgrass PGRs can also be used to reduce scalping and increase ball roll speed. As a result, turfgrass PGRs can reduce costs for golf courses, sport stadiums, and roadside turfgrass management by reducing costs for labor, equipment and fuel.

Several PGRs are currently used by the turfgrass industry. Mefluidide®, Embark Plant Growth Regulator, is a product of PBI/Gordon Corporation (Kansas City, Mo.) that was developed in the later 1970s. Mefluidide® is a PGR that is absorbed by leaves and slows cell division. Flurprimidol®, Cutless, is a product of SePRO Corporation (Carmel, Ind.) that was commercialized in the 1980s. Paclobutrazol®, Trimmit 2SC, is a product of Syngenta Crop Protection Inc. (Greensboro, N.C.) that was also commercialized in the 1980 by The Scotts Company (Marysville, Ohio) with the trade name of TGR Turfgrass Enhancer. Both flurprimidol and paclobutrazol are root absorbed and inhibit the formation of gibberellins during the early stages of the biosynthesis pathway. Trinexapac-ethyl is another product of Syngenta Crop Protection Inc. (Greensboro, N.C.) with trade name of Primo Maxx® that was developed in the 1990s. Trinexapac-ethyl is absorbed by leaves and inhibits the conversion of GA₂₀ to GA₁.

There are several problems associated with commercial turfgrass PGR products. Phytotoxicity is a major factor limiting turfgrass PGRs application, especially in fine turfgrass. Leaf yellowing and damage usually happen after the application of Embark, Cutless or Trimmit. Primo Maxx® was the first PGR to suppress growth as well as improve turfgrass quality (Shepard, D. Turfgrass Trends. April 2002). However, leaf yellowing occurs in the initial state after application. Phytotoxicity can be alleviated by reducing application rate and increasing application frequency. However, this practice increases the labor and equipment cost of PGR application.

A second problem is the different reaction among turfgrass species to PGRs. The effect of PGRs on turfgrass varies with species, varieties, and mowing height (see label of each product). Primo Maxx® is an effective PGR that inhibits almost all the major turfgrass species. However, the rate required to inhibit growth varies in different turfgrass species and with mowing height. When several species or varieties are planted in the same area, this characteristic may cause a decline in the uniformity of turfgrass and thus a decline of turfgrass quality.

Finally, continuous application of turfgrass PGRs may cause abnormalities of physiological metabolism due to the deficiency of gibberellin in plants. Turfgrass that received frequent treatment with gibberellin synthesis inhibitors showed low quality and was susceptible to stresses.

Thus, there is a need to provide a more effective method of turfgrass control that provides faster growth inhibition, provides more growth inhibition, extends the growth inhibitory effect of gibberellin synthesis inhibitors and increases water conservation with respect to turfgrass.

### SUMMARY OF THE INVENTION

The present invention is directed to the treatment of turfgrass with combinations of gibberellin biosynthesis inhibitors (gibberellin synthesis inhibitors) and ABA or its salts. This treatment accelerates growth inhibition provides additional growth inhibition, and extends the growth inhibitory effect of gibberellin synthesis inhibitors. The combination of gibberellin synthesis inhibitors with ABA also decreases evaportranspiration rate and thus reduces water use amount.

Cool season species, such as creeping bentgrass, Kentucky bluegrass and tall fescue, show significant and long lasting growth inhibitory effect to combinations of gibberellin synthesis inhibitors and ABA. However, warm season grasses such as Bermudagrass are not as sensitive as cool season grasses to the combination of gibberellin synthesis inhibitors and ABA.

The present invention provides additional benefit compared to turfgrass PGRs in the current turfgrass market. This invention can be used to enhance gibberellin synthesis inhibitors by producing new formulations or tank mixing ABA with current commercial turfgrass PGRs to inhibit turfgrass growth as well as to reduce water use amount.

This invention can be used to enhance growth control and water use in other monocotyledonous plants as well as dicotyledonous plants.

### DETAILED DESCRIPTION OF THE INVENTION

The present invention inhibits growth of, and decreases water use with, turfgrass. The treatment comprises applying effective, but non-phytotoxic amounts of the S-abscisic acid (ABA; CAS no. 21293-29-8) or its salts in combination with gibberellin biosynthesis inhibitors.

As used herein, the term “salt” refers to the water soluble salts of ABA or ABA analogs or derivatives, as appropriate. Representative such salts include inorganic salts such as the ammonium, lithium, sodium, potassium, calcium and magnesium salts and organic amine salts such as the triethanolamine, dimethylethanolamine and ethanolamine salts.

Gibberellin biosynthesis inhibitors useful in the present invention include, but are not limited to, trinexapac-ethyl, paclobutrazol, uniconazole-P, chlormequat-Cl, mepiquat-Cl, AMO-1618, tetcyclacis, ancymidol, flurprimidol, prohexadione-Ca, daminozide, 16,17-Dihydro Gas, and chlorpropham.

Surfactants can be added to the gibberellin biosynthesis inhibitor ABA solution to improve the performance of the PGRs.

The presently preferred surfactant for ABA performance is Brij 98 (polyoxyethylene (20) oleyl ether) available from Uniqema (Castle, Del.). Other surfactants are also useful in the present invention, including but not limited to, other surfactants in the Brij family (polyoxyethylene fatty alcohol ether) from Uniqema (Castle, Del.), surfactants in the Tween family (Polyoxyethylene sorbitan esters) from Uniqema (Castle, Del.), Silwet family (Organosilicone) from GE Silicones (Wilton, Conn.), Triton family (Octylphenol ethoxylate) from The Dow Chemical Company (Midland, Mich.), Tomadol family (ethoxylated linear alcohol) from Tomah3 Products, Inc. (Milton, Wis.), Myrj family (Polyoxyethylene (POE) fatty acid esters) from Uniqema (Castle, Del.), Span family (Sorbitan ester) from Uniqema (Castle, Del.), and Trylox family (Ethoxylated Sorbitol and Ethoxylated Sorbitol Esters) from Cognis Corporation (Cincinnati, Ohio) as well as commercial surfactant Latron B-1956 (77.0% modified phthalic/glycerol alkyl resin and 23.0% Butyl alcohol) from Rohm & Haas (Philadelphia, Pa.), Capsil (Blend of Polyether-polymethylsiloxanecopolymer and nonionic surfactant) from Aquatrols (Paulsboro, N.J.), Agral 90 (Nonyl phenol ethoxylate) from Norac Concept. Inc. (Orleans, Ontario, Canada), Kinetic (99.00% Proprietary blend of polyalkyleneoxide modified polydimethylsiloxane and nonionic surfactants) from Setre Chemical Company (Memphis, Tenn.), and Regulaid (90.6% 2-butoxyethanol, poloxalene, monopropylene glycol) from KALO, Inc. (Overland Park, Kans.).

Other additives that can be added to the gibberellin biosynthesis inhibitor ABA combination include, but are not limited to, urea, nitrate salts such as ammonium nitrate, humectants such as poly(ethylene glycol) and vegetable oils such as soybean oil, corn oil, cotton oil and palm oil.

This combination of ABA and gibberellin synthesis inhibitors can be used as a formulated liquid or solid product, or as a tank mix. This combination was found to be particularly effective on cool season grasses; other turfgrass species and other plant species are expected to respond similarly. Also, while three gibberellin synthesis inhibitors were tested (trinexapac-ethyl, paclobutrazol and uniconazole-P), other gibberellin synthesis inhibitors are also expected to be effective for the same use.

While the target plants are cool-season turfgrass, other plant species such as bedding plants or vegetable seedlings may also show similar effects.

Depending on the species of turfgrass, mowing height, and environmental conditions, the applied concentration of ABA can vary within wide ranges and is generally in the range of about 0.1 ppm to about 2000 ppm, preferably from about 1 to about 1000 ppm.

Depending on the species of turfgrass, mowing height, environmental conditions, and chemical characteristics of the gibberellin synthesis inhibitor, the applied concentration of the gibberellin synthesis inhibitor can vary within wide ranges and is generally in the range of about 0.1 ppm to about 10,000 ppm, preferably from about 1 ppm to about 1000 ppm.

The water solution may also contain between about 0.01% to about 0.5% v/v surfactants such as Tween 20 (Sigma-Aldrich, St. Louis, Mo.). Water is used as the carrier solvent.

The effective concentration range of active ingredients may vary depending on the water volume applied to grasses as well as other factors such as the plant height, age of the grass, and the requirements of duration of growth inhibition and quality.

The concentration ranges of ABA alone or the combinations of ABA with gibberellin synthesis inhibitors include in principle any concentration range useful for inhibiting turfgrass growth and reducing water use.

The invention can be illustrated by following representative examples.

### EXAMPLES

Greenhouse studies were conducted at the Research Farm of Valent BioSciences Corporation (Long Grove, Ill.). Grasses were grown in pots (18 cm in diameter and 18 cm in height) filled with Promix BX (available from Premier Horticulture Inc. Quakertown, Pa.). Grass was irrigated daily by an overhead irrigation system. The irrigation system was set up with multiple Tornado Mist Spray Heads (10 GPH at 40 PSI-Wetted diameter, NDS/Raindrip, Woodland Hills, Calif.). Spray heads were 1-meter apart from each other and 75 cm above grass canopy. Grass was cut with a scissor at 2.5 cm height and fertilizer (1 g/L all purpose fertilizer 20-20-20, available from The Scotts Company, Marysville, Ohio) was applied once per week.

Field studies were conducted at the nursery green or the practice green at Countryside golf course (Mundelein, Ill.). Both greens were sand based and growing Penncross creeping bentgrass. Grass was managed with typical Illinois golf course management practices.

Chemical solutions were prepared with distilled water. Tween 20 (0.05% v/v) was used as an, adjuvant. Both trinexapac-ethyl (commercial product Primo Maxx, 11.3% active ingredient) and paclobutrazol (commercial product Trimmit 2SC, 22.3% active ingredient) were purchased from Syngenta Crop Protection Inc. (Greensboro, N.C.). Uniconazole-P (commercial product Sumagic, 0.55% active ingredient) was obtained from Valent U.S.A. Corporation (Walnut Creek, Calif.). ABA (90% or 95% active ingredient) was obtained from Lomon BioTechnology Co., Ltd. (Shichuan, China).

Chemical solutions were foliar applied to the turfgrass canopy at the rate of 4-gallons/1000 square feet (or 0.163 L/m⁻²) immediately after finishing the preparation of solutions. When treated with paclobutrazol or uniconazole-P, turfgrass received irrigation within 24 hours after application to flush chemicals to the root zone. After treatment, turfgrasses were arranged in a randomized complete block experimental design. Turfgrass quality, turfgrass height or clip fresh weight was measured on assigned dates. Turfgrass quality was visually rated on a 0-9 scale based on the color, uniformity and density of the grass with 0 as the worst and 9 as the best. Turfgrass height was measured as the distance between canopy surface and soil. Clips were collected from each plot; all plots were cut to a uniform height.

All experiments were randomized complete block experimental design. Data were analyzed by analysis of variance. Duncan's new multiple range tests at α=0.05 were used for mean separations.

### Example 1

Kentucky bluegrass sod (unknown variety) was purchased from Deak sod farms, Inc. (Union Grove, Wis.). Grass was grown in the greenhouse in pots (n=6 pots per treatment) for establishment before treatment. One time foliar applications were made with trinexapac-ethyl alone (40, 80, and 160 ppm) or in combination with ABA (200 ppm). Turfgrass quality was evaluated at 7 days after treatment, and turfgrass height was measured at 7 and 23 days after treatment. Tween 20 (0.05% w/v) was included as an adjuvant. Turfgrass canopy was not cut during the experimental period.

At 7 days after treatment trinexapac-ethyl (40, 80 or 160 ppm), ABA (200 ppm), and their combinations reduced turfgrass heights (Table 1). Combination of ABA with 40, 80 or 160-ppm trinexapac-ethyl was better than any trinexapac-ethyl treatment alone at 7 days after treatment. By 23 days after treatment, ABA did not control turfgrass heights. Surprisingly, at 23 days after treatment, the combinations of ABA with 40, 80 or 160 ppm trinexapac-ethyl was better than 40, 80 or 160 ppm trinexapac-ethyl alone thus suggesting a synergistic effect between ABA and trinexapac-ethyl.

At 23 days after treatment, turfgrass quality was the same for the combination of ABA with trinexapac-ethyl than for trinexapac-ethyl alone at any rate (Table 1).

TABLE 1
Effect of ABA, trinexapac-ethyl, and combinations
on height and quality of Kentucky bluegrass.

| | Turfgrass height (cm) | | Quality |
|---|---|---|---|
| | 7 days after | 23 days after | 23 days after |
| Treatment | treatment | treatment | treatment |
| Control | 12.0 | 18.8 | 8.0 |
| 40 ppm trinexapac-ethyl | 9.5 | 18.0 | 7.8 |
| 80 ppm trinexapac-ethyl | 7.4 | 17.3 | 8.0 |
| 160 ppm trinexapac-ethyl | 6.1 | 13.3 | 7.0 |
| 200 ppm ABA | 9.4 | 19.0 | 8.0 |
| 200 ppm ABA + 40 ppm | 6.1 | 15.5 | 8.0 |
| trinexapac-ethyl | | | |
| 200 ppm ABA + 80 ppm | 5.4 | 14.8 | 7.3 |
| trinexapac-ethyl | | | |
| 200 ppm ABA + 160 ppm | 5.3 | 12.3 | 6.8 |
| trinexapac-ethyl | | | |

### Example 2

Kentucky bluegrass sod (unknown variety) was purchased from Deak sod farms, Inc. (Union Grove, Wis.). Grass was grown in the greenhouse in pots (n=6 pots per treatment) for establishment before treatment. One time foliar applications were made with paclobutrazol alone (5, 50 and 50.0 ppm paclobutrazol applied as Trimmit 2SC) or in combination with ABA (200 ppm). Turfgrass quality was evaluated at 7 days after treatment, and turfgrass height was measured at 7 and 28 days after treatment. Tween 20 (0.05% w/v) was included as an adjuvant. The turfgrass canopy was cut every 7 days during the experimental period.

At 7 days after treatment, 50 or 500 ppm paclobutrazol did not reduce turfgrass height and ABA (200 ppm) reduced growth only slightly compared to the control (Table 2). However, the combination of ABA with either rate of paclobutrazol reduced height substantially thus suggesting synergistic activity. At 28 days after treatment, the height of ABA treated turfgrass was slightly greater than the control suggesting that the ABA treatment was no longer effective. Surprisingly, the combination treatments of ABA with paclobutrazol controlled growth more than paclobutrazol alone at either rate.

At 7 days after treatment, addition of ABA to paclobutrazol did not reduce turfgrass quality compared to paclobutrazol alone at either rate (Table 2).

TABLE 2
Effect of ABA, paclobutrazol, and combinations
on height and quality of Kentucky bluegrass.

| | | | Turfgrass |
|---|---|---|---|
| | Turfgrass height (cm) | | quality |
| | 7 days after | 28 days after | 7 days after |
| Treatment | treatment | treatment | treatment |
| 0 ppm paclobutrazol | 13.0 | 9.4 | 8.0 |
| 50 ppm paclobutrazol | 12.3 | 8.6 | 8.0 |
| 500 ppm paclobutrazol | 12.1 | 6.2 | 7.1 |
| 200 ppm ABA | 11.7 | 9.7 | 8.0 |
| 200 ppm ABA + 50 ppm | 11.0 | 7.9 | 8.0 |
| paclobutrazol | | | |
| 200 ppm ABA + 500 ppm | 9.8 | 6.1 | 7.8 |
| paclobutrazol | | | |

### Example 3

Kentucky bluegrass sod (unknown variety) was purchased from Deak sod farms, Inc. (Union Grove, Wis.). Grass was grown in the greenhouse in pots (n=6 pots per treatment) for establishment before treatment. One time foliar applications were made with uniconazole-P alone (10 ppm applied as Sumagic) or in combination with ABA (200 ppm). Turfgrass height was measured at 7 and 23 days after treatment. Tween 20 (0.05% w/v) was included as an adjuvant. The turfgrass was cut every 7 days.

At 7 days after treatment uniconazole-P (10 ppm) and ABA (200 ppm) had little effect on turfgrass height compared to the control (Table 3). Combination of ABA with uniconazole-P was better than uniconazole-P treatment. By 42 days after treatment, the height of uniconazole-P and ABA treated turfgrass was greater than the control showing a rebound effect of turfgrass growth. In contrast, surprisingly, the combination of ABA with uniconazole-P was still controlling growth compared to the control. This suggests that ABA and uniconazole worked synergistically to extend growth control.

TABLE 3
Effect of ABA, uniconazole-P, and combinations
on height of Kentucky bluegrass.

| | Turfgrass height (cm) | |
|---|---|---|
| | 7 days after | 42 days after |
| Treatment | treatment | treatment |
| Control | 13.0 | 8.9 |
| 10 ppm uniconazole-P | 12.3 | 9.8 |
| 200 ppm ABA | 12.3 | 9.4 |
| 200 ppm ABA + 10 ppm uniconazole-P | 11.6 | 7.9 |

### Example 4

ABA (200 ppm), trinexapac-ethyl (12.5 or 50 ppm), and their combinations were one time foliar applied to creeping bentgrass in a golf course green. Tween 20 (0.05% w/v) was included as an adjuvant.

At 7 days after treatment, creeping bentgrass heights for either combination treatment ABA (200 ppm) and trinexapac-ethyl (12.5 or 500 ppm) were shorter than for ABA or trinexapac-ethyl treatments alone (Table 4). Although ABA did not control creeping bentgrass growth at 14 days after treatment, turfgrass heights for grass treated with the combinations of ABA with either rate of trinexapac-ethyl were shorter than for trinexapac-ethyl treatment alone.

TABLE 4
Effect of ABA, trinexapac-ethyl, and combinations
on height of creeping bentgrass.

Turfgrass height (cm)

| | 7 days after | 14 days after |
|---|---|---|
| Treatment | treatment | treatment |
| Control | 6.9 | 8.3 |
| 12.5 ppm trinexapac-ethyl | 6.0 | 8.5 |
| 50 ppm trinexapac-ethyl | 4.3 | 5.5 |
| 200 ppm ABA | 5.3 | 8.2 |
| 200 ppm ABA + 12.5 ppm | 3.8 | 7.6 |
| trinexapac-ethyl | | |
| 200 ppm ABA + 50 ppm | 3.5 | 4.8 |
| trinexapac-ethyl | | |

### Example 5

ABA (200 ppm), uniconazole-P (0.5 ppm), and their combination were one time foliar applied to creeping bentgrass in a golf course green. Tween 20 (0.05% w/v) was included as an adjuvant.

The combination of 200 ppm ABA with 0.5 ppm uniconazole-P was more effective at controlling height and clip weight than ABA or 0.5 ppm uniconazole-P alone (Table 5). This ABA/uniconazole-P combination was as effective as using uniconazole-P at 10 times the rate of uniconazole. No effect on turfgrass quality was observed throughout the study (not shown).

TABLE 5
Effect of ABA, uniconazole-P, and combinations on
height and clip weight of creeping bentgrass.

| | Turfgrass height (cm) | | Clip weight (g) | |
|---|---|---|---|---|
| | 7 days | 49 days | 7 days | 49 days |
| | after | after | after | after |
| Treatment | treatment | treatment | treatment | treatment |
| Control | 5.7 | 4.6 | 2.5 | 2.8 |
| 0.5 ppm | 6.0 | 4.8 | 2.4 | 3.3 |
| uniconazole-P | | | | |
| 200 ppm ABA | 5.0 | 4.6 | 2.0 | 2.7 |
| 200 ppm ABA + 0.5 | 5.0 | 4.4 | 1.7 | 2.7 |
| ppm uniconazole-P | | | | |

### Example 6

The growth inhibitory effect of trinexapac-ethyl and its combination with ABA was tested at Kentucky bluegrass (variety Midnight) and Tall fescue (variety K-31) that were grown starting from seed for three months. Trinexapac-ethyl alone (80 or 160 ppm) and their combinations with 200 ppm ABA were one time foliar applied to both species.

The combinations of ABA (200 ppm) with trinexapac-ethyl (80 or 160 ppm) were more effective in controlling either Kentucky bluegrass (7 days after treatment) or tall fescue (7 or 21 days after treatment) than trinexapac-ethyl alone (Table 6).

TABLE 6
Effect of ABA and trinexapac-ethyl combinations
on height of Kentucky bluegrass and tall fescue.

| | Turfgrass height (cm) | | |
|---|---|---|---|
| | Kentucky | | |
| | bluegrass | | |
| | cv Midnight | Tall fescue, cv K-31 | |
| | 7 days after | 7 days after | 21 days after |
| Treatment | treatment | treatment | treatment |
| Control | 8.1 | 12.9 | 24.9 |
| 80 ppm trinexapac-ethyl | 7.6 | 11.8 | 19.8 |
| 160 ppm trinexapac-ethyl | 6.7 | 9.7 | 20.1 |
| 200 ppm ABA + 80 ppm | 6.3 | 8.5 | 19.3 |
| trinexapac-ethyl | | | |
| 200 ppm ABA + 160 ppm | 5.7 | 7.6 | 18.8 |
| trinexapac-ethyl | | | |

### Example 7

ABA (200 ppm) alone did not reduce, but in fact slightly increased growth of Bermudagrass at 7 days after treatment based on clip weight (Table 7). Uniconazole-P (50 ppm) reduced Bermudagrass growth somewhat (14% less clip weight compared to control). However, the combination of ABA and uniconazole-P substantially reduced clip weight (46% less clip weight compared to control). This indicates that the combination of ABA and uniconazole has synergistic growth reduction on Bermudagrass.

TABLE 7
Effect of ABA and uniconazole-P combinations
on clip weight of Bermudagrass

| | Clip weight (g) |
|---|---|
| Treatment | 7 days after treatment |
| Control | 1.5 |
| 50 ppm uniconazole-P | 1.4 |
| 200 ppm ABA | 1.8 |
| 200 ppm ABA + 50 ppm uniconazole-P | 1.1 |

### Example 8

ABA (500 ppm), trinexapac-ethyl (25 ppm), and their combinations were one time foliar applied to creeping bentgrass in a golf course green. Tween 20 (0.05% w/v) was included as an adjuvant.

At 4 days after treatment, soil moisture of the green with treatment ABA (500 ppm) and the combination of ABA with trinexapac-ethyl (25 ppm) was higher than trinexapac-ethyl treatments alone (Table 8). Although ABA did not affect soil moisture and trinexapac-ethyl alone increased soil moisture at 7 days after treatment, the combinations of ABA with trinexapac-ethyl had higher soil moisture than trinexapac-ethyl treatment alone.

TABLE 8
Effect of ABA, trinexapac-ethyl, and combinations
on soil moisture of creeping bentgrass green.

Soil moisture (%)
Days after treatment

| Treatments | 2 | 7 |
|---|---|---|
| Control | 11.3 | 11.0 |
| 25 ppm trinexapac-ethyl | 11.2 | 12.7 |
| 500 ppm ABA | 11.4 | 10.9 |
| 25 ppm trinexapac-ethyl + 500 ppm ABA | 11.4 | 12.9 |

### Example 9

The effect of ABA and trinexapac-ethyl combinations on transpiration and growth inhibition of dicotyledonous (tomato) was also examined in the greenhouse condition. Tomato (variety: Rutgers) seeds were sown in 18-cell flat filled with Promix PGX (available from Premier Horticulture Inc. Quakertown, Pa.) and grown for 3 weeks to allow for germination and initial growth. Plants were then transplanted into pots (18 cm in diameter and 18 cm in height), filled with Promix BX (available from Premier Horticulture Inc. Quakertown, Pa.), and grown for one week before the chemical treatment. Plants received daily irrigation and weekly fertilizer (1 g/L all purpose fertilizer 20-20-20, available from The Scotts Company, Marysville, Ohio).

During the chemical treatment, a 15 mL (2.5 mL/plant) solution was foliar sprayed on the tomato canopy. Leaf transpiration rates were measured using a LI-1600 Steady State Porometer (LI-Cor, Lincoln, Nebr.) at 1, 2, 3, 4, 7, 10 and 15 days after treatment. Leaf transpiration rate was normalized to the percentage of control plant to minimize the experimental errors caused by environmental factors. Plant height was measured at 0, 2, 4, 7, 10 and 15 days after treatment. Growth rate was calculated based on the changes of plant height in certain intervals. The plants were harvested and the leaf number was counted at 15 days after treatment.

ABA inhibited tomato leaf transpiration in a dose-dependent manner in the first 7 days after treatment. Higher concentration of ABA inhibited more transpiration than low concentration (Table 9). Trinexapac-ethyl had little effect on transpiration and was not correlated to trinexapac-ethyl concentrations. The combination of ABA and trinexapac-ethyl inhibited much more transpiration than ABA alone or trinexapac-ethyl alone at same rate. This transpiration inhibition also lasted longer than ABA alone or trinexapac-ethyl alone the same rate.

TABLE 9
Effect of ABA, trinexapac-ethyl, and their combinations
on tomato leaf transpiration inhibition

| | Transpiration (% of control) | | | | | | |
|---|---|---|---|---|---|---|---|
| | Days after treatment | | | | | | |
| Treatment | 1 | 2 | 3 | 4 | 7 | 10 | 15 |
| Control | 100 | 100 | 100 | 100 | 100 | 100 | 100 |
| 250 ppm ABA | 87 | 86 | 91 | 95 | 99 | 98 | 99 |
| 500 ppm ABA | 78 | 78 | 84 | 88 | 98 | 99 | 99 |
| 1000 ppm ABA | 60 | 73 | 76 | 84 | 93 | 99 | 97 |
| 2000 ppm ABA | 46 | 58 | 70 | 76 | 85 | 95 | 100 |
| 250 ppm trinexapac-ethyl | 91 | 99 | 91 | 95 | 100 | 98 | 98 |
| 500 ppm trinexapac-ethyl | 91 | 95 | 89 | 96 | 100 | 99 | 100 |
| 1000 ppm trinexapac-ethyl | 92 | 96 | 91 | 98 | 100 | 103 | 99 |
| 2000 ppm trinexapac-ethyl | 95 | 94 | 90 | 98 | 97 | 99 | 98 |
| 250 ppm ABA + 250 ppm trinexapac-ethyl | 71 | 73 | 84 | 88 | 96 | 98 | 97 |
| 500 ppm ABA + 500 ppm trinexapac-ethyl | 55 | 62 | 73 | 79 | 95 | 97 | 99 |
| 1000 ppm ABA + 1000 ppm trinexapac-ethyl | 20 | 45 | 57 | 71 | 84 | 92 | 98 |
| 2000 ppm ABA + 2000 ppm trinexapac-ethyl | 4 | 10 | 25 | 30 | 70 | 86 | 98 |

ABA decreased tomato plant height in a dose dependent manner (Table 10). High concentration of ABA caused lower plant height than low concentration. The growth inhibition by high concentration ABA also lasted longer than low concentration ABA. Trinexapac-ethyl decreased plant height at 7 days after treatment but increased plant height at 15 days after treatment. In the first 7 days (for 250 or 500 ppm) or 10 days (for 1000 or 2000 ppm) after treatment, tomato plants treated with ABA and trinexapac-ethyl combination were shorter than plants treated with ABA alone or trinexapac-ethyl alone at same rate.

TABLE 10
Effect of ABA, trinexapac-ethyl, and their
combinations on tomato plant height

| | Plant height (cm) | | | | | |
|---|---|---|---|---|---|---|
| | Days after treatment | | | | | |
| Treatment | 0 | 2 | 4 | 7 | 10 | 15 |
| Control | 10.2 | 14.1 | 17.0 | 29.1 | 30.3 | 45.1 |
| 250 ppm ABA | 9.9 | 12.8 | 16.3 | 28.5 | 30.4 | 45.5 |
| 500 ppm ABA | 10.1 | 12.5 | 16.3 | 27.0 | 29.6 | 44.1 |
| 1000 ppm ABA | 9.8 | 11.7 | 14.5 | 25.3 | 27.4 | 41.6 |
| 2000 ppm ABA | 9.8 | 11.5 | 14.9 | 23.2 | 25.8 | 40.5 |
| 250 ppm trinexapac-ethyl | 10.3 | 13.6 | 16.4 | 28.7 | 32.3 | 50.6 |
| 500 ppm trinexapac-ethyl | 9.8 | 13.1 | 15.9 | 28.3 | 31.2 | 50.7 |
| 1000 ppm trinexapac-ethyl | 10.0 | 12.3 | 15.4 | 26.7 | 31.3 | 52.7 |
| 2000 ppm trinexapac-ethyl | 9.9 | 12.2 | 15.8 | 23.9 | 29.3 | 50.6 |
| 250 ppm ABA + 250 ppm | 10.1 | 12.7 | 16.6 | 27.9 | 34.7 | 49.7 |
| trinexapac-ethyl | | | | | | |
| 500 ppm ABA + 500 ppm | 9.8 | 12.0 | 14.4 | 26.8 | 32.2 | 50.8 |
| trinexapac-ethyl | | | | | | |
| 1000 ppm ABA + 1000 ppm | 9.8 | 11.5 | 12.8 | 23.7 | 28.3 | 47.7 |
| trinexapac-ethyl | | | | | | |
| 2000 ppm ABA + 2000 ppm | 10.0 | 10.9 | 11.4 | 17.5 | 22.5 | 42.8 |
| trinexapac-ethyl | | | | | | |

ABA and trinexapac-ethyl decreased tomato growth rate (plant height) in a dose-dependent manner during the experimental period (Table 11). Trinexapac-ethyl decreased tomato growth rate during the first 7 days after treatment and then increased growth rate at 15 days after treatment (Table 11). The combination of ABA and trinexapac-ethyl decreased growth rate more than ABA or trinexapac-ethyl alone at same rate.

TABLE 11
Effect of ABA, trinexapac-ethyl, and their
combinations on tomato growth rate

| | Growth rate (cm day⁻¹) | | | | |
|---|---|---|---|---|---|
| | Days after treatment | | | | |
| Treatment | 2 | 4 | 7 | 10 | 15 |
| Control | 2.0 | 1.7 | 2.7 | 2.0 | 2.3 |
| 250 ppm ABA | 1.5 | 1.6 | 2.7 | 2.1 | 2.3 |
| 500 ppm ABA | 1.3 | 1.6 | 2.4 | 2.0 | 2.3 |
| 1000 ppm ABA | 1.0 | 1.2 | 2.2 | 1.8 | 2.0 |
| 2000 ppm ABA | 0.9 | 1.3 | 1.9 | 1.6 | 1.9 |
| 250 ppm trinexapac-ethyl | 1.7 | 1.5 | 2.6 | 2.2 | 2.7 |
| 500 ppm trinexapac-ethyl | 1.7 | 1.5 | 2.6 | 2.2 | 2.6 |
| 1000 ppm trinexapac-ethyl | 1.2 | 1.4 | 2.4 | 2.1 | 2.8 |
| 2000 ppm trinexapac-ethyl | 1.2 | 1.5 | 2.0 | 2.0 | 2.7 |
| 250 ppm ABA + 250 ppm trinexapac-ethyl | 1.3 | 1.6 | 2.6 | 2.5 | 2.6 |
| 500 ppm ABA + 500 ppm trinexapac-ethyl | 1.1 | 1.1 | 2.4 | 2.3 | 2.7 |
| 1000 ppm ABA + 1000 ppm trinexapac-ethyl | 0.9 | 0.8 | 2.0 | 1.9 | 2.5 |
| 2000 ppm ABA + 2000 ppm trinexapac-ethyl | 0.4 | 0.4 | 1.1 | 1.3 | 2.0 |

ABA alone at any concentrations, trinexapac-ethyl alone at any concentrations, and the ABA and trinexapac-ethyl combination at 1000 ppm each or below, did not significantly decreased tomato leaf number. Only the combination of 2000-ppm ABA and 2000 ppm-trinexapac-ethyl decreased leaf number (Table 12).

TABLE 12
Effect of ABA, trinexapac-ethyl, and their
combinations on tomato leaf number

| | Leaf number |
|---|---|
| Treatment | 15 days after treatment |
| Control | 12.0 |
| 250 ppm ABA | 12.2 |
| 500 ppm ABA | 12.0 |
| 1000 ppm ABA | 12.0 |
| 2000 ppm ABA | 12.0 |
| 250 ppm trinexapac-ethyl | 12.0 |
| 500 ppm trinexapac-ethyl | 11.7 |
| 1000 ppm trinexapac-ethyl | 11.7 |
| 2000 ppm trinexapac-ethyl | 11.5 |
| 250 ppm ABA + 250 ppm trinexapac-ethyl | 11.8 |
| 500 ppm ABA + 500 ppm trinexapac-ethyl | 11.8 |
| 1000 ppm ABA + 1000 ppm trinexapac-ethyl | 11.7 |
| 2000 ppm ABA + 2000 ppm trinexapac-ethyl | 11.0 |

1. A method of accelerating and extending the growth inhibitory effect of gibberellin synthesis inhibitors that comprises applying to said inhibitors an effective amount of S-abscisic acid or a salt thereof.

2. The method of claim 1 wherein the gibberellin synthesis inhibitor is trinexapac-ethyl.

3. The method of claim 1 wherein the gibberellin synthesis inhibitor is paclobutrazol.

4. A method of claim 1 wherein the gibberellin synthesis inhibitor is uniconazole-P.

5. A method of improving the reduction in soil moisture caused by gibberellin synthesis inhibitors that comprises applying an effective amount of S-abscisic acid or a salt thereof to the soil.

6. The method of claim 2 wherein the trinexapac-ethyl and S-abscisic acid is applied to Kentucky bluegrass, creeping bentgrass, tall fescue or Bermudagrass.

7. The method of claim 2 wherein the trinexapac-ethyl and S-abscisic acid is applied to dicotyledonous plants.
